(19) United States
(12) Patent Application Publication
(10) Pub. No.: US 2008/0058647 A1
(43) Pub. Date: Mar. 6, 2008

(54) Means for Performing Measurements in a Vessel

(75) Inventors: Sascha Kruger, Hamburg (DE); Jorn Borgert, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V.

(21) Appl. No.: 10/565,933

(22) PCT Filed: Jul. 13, 2004
(86) PCT No.: PCT/IB04/51207

§ 371 Date: Jan. 20, 2006

(30) Foreign Application Priority Data
Jul. 25, 2003 (EP) 03102292.4

(51) Int. Cl. A61B 8/06; A61B 5/026; A61B 8/12

(52) U.S. Cl. 600/454; 600/468; 600/504

(57) ABSTRACT

The invention relates to a facility that can be used, in particular, to measure the flow conditions in a blood vessel. The facility comprises a catheter (16) having a bundle (15) of optical waveguides that connects control and measurement facilities (20) outside the body with an optical unit (10) at the catheter tip. The light (λ_{K}) generated by a cavitation light laser source (30) is beamed via the catheter (16) and the optical unit (10) into a focus region (2) in the vessel lumen, where it generates cavitation bubbles (3). The movement of the cavitation bubbles (3) with the blood flow is determined by the particle-measuring unit (20) that is based, for example, on phase-Doppler anemometry and/or the Doppler shift. As a result of suitable design of the optical unit (10), the focus region (2) can be displaced as desired radially and rotationally inside the vessel so that a vessel cross section can be scanned in a spatially resolved way. Furthermore, a spectral analysis of the light arriving from the focus region (2) is possible in order, for example, to analyze the chemical composition in this region. The reaching of the vessel wall (1) can be detected by the moving focus region (2) and used for a vessel measurement and/or to switch off the cavitation light laser (30).

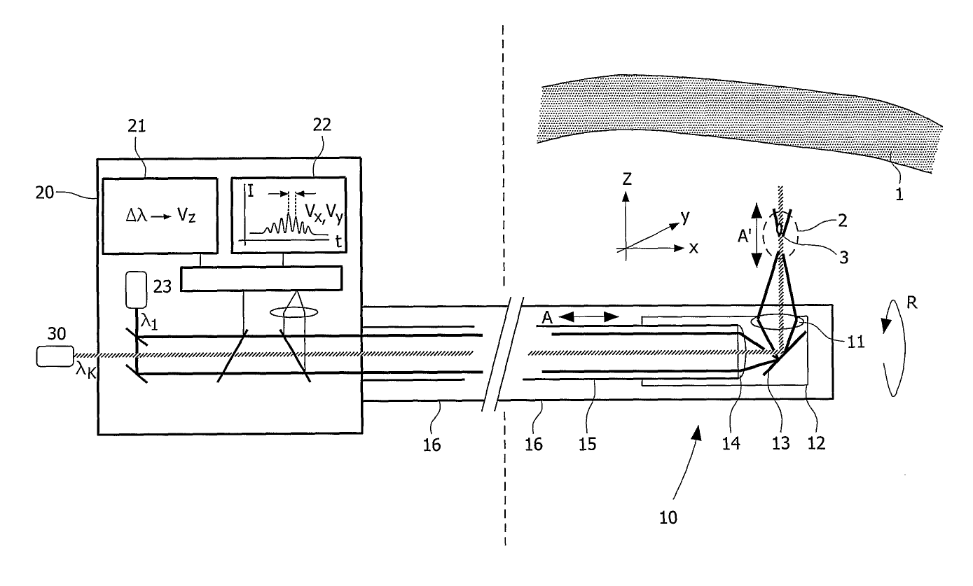

The invention relates to various means for performing measurements in a vessel or another environment. In particular, it relates to a device and a method for measuring flow in a fluid, a facility for invasive interventions with a catheter and a method for detecting the position of a vessel wall.

In the framework of minimal invasive surgery, it is necessary to execute measurements and interventions of various types with the aid of a catheter in a vessel system. In this connection, the analysis of the intraluminal (blood) flow in a vessel, in particular, is of great diagnostic importance. In the treatment of coronary vessels, measurement of the blood flow with high spatial and temporal resolution could supply important information items relating to the functional efficiency of the coronary vessels and, in addition, indicate potential risks of the formation of deposits. In conjunction with purely anatomically imaging modalities, such as, for example, computer tomography, a flow measurement can supply important additional information items and help to prevent incorrect interpretations due to artifacts or ambiguous information items. For example, a flow measurement after completing a percutaneous transluminal coronary angioplasty (PTCA) can make it possible to check the success of the positioning of a stent at the point of a stenosis.

Known methods for determining blood flow are based, inter alia, on the measurement of the Doppler shift that occurs at a moving particle during reflection of an ultrasonic wave or a laser-light pulse. The spatial resolution of such methods is, however, relatively low and, as a rule, they cannot detect a plurality of components of the flow velocity at the same time. Furthermore, the literature (D. Lipsch, A. Poll, G. Pflugbeil: “In vitro laser anemometry blood flow systems”, Proceedings of the Society of Photo-Optical Instrumentation, Vol. 2052, pages 163-178, 1993) discloses the application of so-called “phase-Doppler anemometry” for determining blood flow, but this method is regarded as unsuitable for in vivo measurements.

As already mentioned, the particle-measuring unit may be based on any measurement principle suitable for determining the movement of particles. Preferably, the particle-measuring unit is designed in this regard to measure particle movement with the aid of phase-Doppler anemometry and/or a Doppler shift. Reference is made to the relevant literature (for example, W. D. Bachalo, M. I. Houser: “Phase-Doppler-Spray Analyzer for simultaneous measurements of drop size and velocity distributions, Opt. Engineering 23, pages 583-590) for the known details of this measurement method. For phase-Doppler anemometry, a particle-measuring unit needs in this connection, for example, at least one (laser) light source, focusing optics for the interfering superimposition of two beams from the light source in a focus region, a measuring facility for detecting the light scattered at particles in the focus region and a unit for analyzing and evaluating intensity attenuations of the measured scattered light.

In accordance with another configuration, the particle-measuring unit may be designed to determine the particle movement from the detection of light that is emitted by the moving particles. Light-emitting particles may, for example, be observed with conventional imaging optics, with the result that their movement can be investigated by standard methods of image analysis. Such a particle-measuring unit would make it possible to utilize the effect of the sonoluminescence of cavitation bubbles, that is to say of the light emission induced by cavitation.

The invention furthermore relates to a facility for invasive interventions of a diagnostic and/or a therapeutic type, which facility contains a catheter. The catheter has in this connection an optical unit disposed at the catheter tip that is to be introduced into the vessel system of a patient. The optical unit is designed to receive light selectively from a focus region situated outside the catheter and/or, conversely, to beam light into the focus area. Furthermore, the optical unit is designed in such a way that the radial position of the focus region relative to the catheter can be externally adjusted. The term “radial” relates in this connection to the longitudinal axis of the catheter.

In the case of the facility described, it is possible for a user of the catheter to alter the position of the focus area of the optical unit from the outside without moving the catheter during an invasive intervention. In this connection, the focus area, in particular a vessel in which the catheter tip is situated, can continuously move through in the radial direction so that measurements and/or manipulations can be executed in the focus region at various spatial positions in the vessel. Various applications of this possibility are explained below in connection with embodiments of the facility.

In accordance with a first embodiment of the facility, the optical unit is constructed so as to be rotatable around the catheter axis relative to the catheter. The focus region can therefore be rotated around the catheter tip by rotating the optical unit in order to make possible measurements and/or manipulations at various points. By combining the radial and the rotary displacements of the focus region, it is furthermore possible, for example, to scan a sectional area through a vessel on a spiral path.

In accordance with a further embodiment of the facility, the catheter contains a bundle comprising at least one optical waveguide that connects the optical unit to the start of the catheter (which remains, according to definition, outside the body). Light can be guided to the optical unit from outside via the optical waveguides and focused therefrom in the focus region. Conversely, (only) the light received from the focus region via the optical unit can be selectively fed to the optical waveguides and from the latter to the outside. This makes it possible to dispose light sources or appliances for light analysis outside the body. Furthermore, the bundle of optical waveguides simultaneously makes a mechanical connection of the optical unit to the outside region so that, for example, the optical unit can be adjusted by means of an axial and/or rotary movement of the optical waveguide relative to the catheter.

In accordance with a preferred configuration of the facility, the latter has a scanning unit that is designed to vary the position of the focus region systematically by a suitable adjustment of the optical unit and, furthermore, to analyze light picked up by the optical unit from the respective current focus region in regard to characteristic properties of the focus region. With the aid of the scanning unit, which may contain, in particular, a data-processing facility for control and evaluation, the space around the optical unit can therefore be systematically scanned, information items being obtained from each focusing region of the optical unit with high spatial resolution. This makes possible, for example, a structural analysis of the vessel lumen in which, in particular, the position of the vessel wall can be determined from the qualitative change occurring at that point in the light picked up from the focus region. Furthermore, the light arriving from the focus region can also yield conclusions relating to the molecular composition of the focus region, for example if fluorescent light having a substance-specific wavelength is involved. The scanning unit consequently also makes possible a spatially resolved molecular analysis of a vessel lumen. In combination with the structural analysis, in particular, the effect of a drug at the vessel wall can be checked in this connection.

Preferably, the facility comprises a spectrometer that enables light picked up from the focus region of the optical unit to be analyzed spectrally. In this connection, the spectrum may yield, for example, important information items relating to the material composition and/or relating to movement processes (Doppler shift) in the focus region.

In accordance with another configuration, the facility contains a particle-measuring unit that is designed to generate a modulated light field for phase-Doppler anemometry in the focus region by means of the optical unit. The variable position of the focus region then makes it possible to measure the flow conditions at various points in the vessel with high spatial resolution.

In another embodiment of the facility, the latter contains an activation unit that is designed to inject light via the optical unit into its focus region in order to initiate processes by interaction of the light with the matter situated in the focus area. For example, the light of the activation unit may activate drugs in a controlled manner in certain zones of the vessel (in particular at the vessel wall).

Furthermore, the activation unit may contain a laser source for “cavitation light” that is designed to generate cavitation bubbles in the focus region of the optical unit. As has already been explained, the cavitation bubbles generated with the laser source can be used as particles for determining the flow conditions in the vessel. Preferably, use is made in this connection of a particle-measuring unit of the type described above that is based on phase-Doppler anemometry since, in that case, the optical unit can be used simultaneously for introducing the cavitation light into the focus region and for phase-Doppler anemometry. In order to avoid damage to the tissue by the relatively high-power cavitation light, an automatic suppression of the cavitation light is preferably provided if the focus region leaves the lumen of a vessel and touches the vessel wall or transgresses it. This condition can be monitored, for example, with a scanning unit of the type explained above.

The invention furthermore relates to a method for measuring flow in a fluid in which cavitation bubbles are generated in the fluid and the movement of the cavitation bubbles is observed.

Furthermore, the invention relates to a method for detecting the position of a vessel wall in which light is picked up from a focus region continuously displaced in the vessel and a qualitative change in the light picked up is detected.

The two methods mentioned relate in general form to the steps that can be executed with a device for measuring flow or a facility for invasive intervention of the type explained above. Reference is therefore made to the above description for an explanation of details, advantages and embodiments of the methods. Thus, within the framework of the method for measuring flow, in particular, ultrasound or laser light can be used to generate cavitation bubbles. The cavitation bubbles can be observed, in particular, with the aid of sonoluminescence, phase-Doppler anemometry and/or Doppler shift. The method for detecting the position of a vessel wall can be used to measure the cross section and, if executed at a plurality of axial positions, the spatial configuration of a vessel segment. Furthermore, proceeding from the method, controlled manipulations, such as, for example, the activation of drugs at the vessel wall can also be controlled.

These and other aspects of the invention are apparent from and will be elucidated with reference to the sole FIGURE, which shows diagrammatically a facility according to the invention for measuring flow with the aid of a catheter.

The left-hand part of the FIGURE shows the facilities connected to the beginning of the catheter 16 outside the body, whereas the right-hand part of the FIGURE shows the region of the catheter tip, which is situated in a vessel having the vessel wall 1. In this connection, the FIGURE is very diagrammatic and, in particular, not to scale.

In its interior, the catheter 16 contains a bundle 15 of light guides or optical fibers that is connected to its end situated in the catheter tip by a first lens 14. Said end of the fiber bundle 15 comprising the first lens 14 is disposed in an axially displaceable manner (double arrow A) in the cylindrical casing 12 of an optical unit 10. Furthermore, situated in said casing 12 is a mirror 13 that is inclined with respect to the catheter axis and that reflects light emerging from the fiber bundle 15 through the lens 14 to the side (that is to say radially with respect to the catheter axis). A second lens 11 disposed in the circumferential wall of the housing 12 focuses the light arriving from the mirror 13 in a focus region 2, which is situated outside the catheter 16 in the lumen of the vessel and which involves a small spatial volume of typically 10 to 50 μm diameter. The light path described is, of course, reversible so that light generated by scattering, emission or other processes in the focus region 2 is picked up by the optical unit 10 and conveyed into the fiber bundle 15.

As already mentioned, the fiber bundle 15 is axially displaceable relative to the housing 12 of the optical unit 10. The position of the focus region 2 can be moved in a controlled way radially (double arrow A′) by such a displacement (double arrow A).

Furthermore, the casing 12 of the optical unit 10 and the fiber bundle 15 are mounted so as to be rotatable relative to the catheter 16 around its axis, the casing 12 and the fiber bundle 15 being coupled in a rotation-locked manner to one another. As a result of a rotation of the fiber bundle 15 impressed externally, the latter consequently drives the housing 12 as a result of which the focus region 2 can be rotated around the catheter axis as desired (arrow R).

As a result of a combined axial movement (A) and rotary movement (R) of the fiber bundle 15, the focus region 2 can scan a cross-sectional plane extending through the vessel on a spiral path. As a result of an axial advance of the entire catheter 16, the cross-sectional area can at the same time be positioned as desired along the axis of the vessel so that, as a result, a three-dimensional scanning of the vessel by the focus region 2 is possible. Manipulations and/or measurements taking place in the focus region 2 can consequently be undertaken in a positionally resolved manner at any position in the vessel.

A possible application of the above-described arrangement is the measurement of flow conditions in the blood vessel. In this process, the flow is measured by means of observing the cavitation bubbles 3 that are moved in accordance with the local flow velocity. The cavitation bubbles 3 are generated by “cavitation light” λ_{K} of a high-power laser 30 that is disposed outside the body and whose cavitation light λ_{K} is beamed via the optical fiber bundle 15, the first lens 14, the mirror 13 and the second lens 11 into the focus region 2. In the focus region, the cavitation light then generates cavities (small cavitation bubbles 3) as a result of liquid evaporation, reference being made to the relevant literature (for example I. Akhatov, O. Lindau, A. Topolnikov, R. Mettin, N. Vakhitova, W. Lauterborn: “Collapse and Rebound of a Laser-Induced Cavitation bubble” 2001, Physics of Fluids 13(10), pages 2805-2819) for the detailed description of the basic processes.

The cavitation bubbles are essentially generated in the center of the focus region 2 and then convectively entrained by the flow of the blood. In the case of the facility shown comprising a particle-measuring unit, this movement is observed and is based on the principles of phase-Doppler anemometry (PDA) and the Doppler shift in order to determine the velocity components in all three spatial directions x, y and z. For PDA, a stationary light field having regular spatial amplitude modulations is generated in the focus region 2. This is done by means of the interference of two laser light beams of wavelength λ₁ that are generated by a laser source 23 and are beamed via the fiber bundle 15, the first lens 14, the mirror 13 and the second lens 11 at various angles into the focus region 2. In the focus region 2, interference of the two beams then occurs and results in the desired intensity modulations in a spatial direction (for example, x). In a similar way, the light of a second laser (not shown) having another wavelength λ₂ is brought into the focus region for the purpose of self-interference, but the resultant variation takes place in another spatial direction (for example, y). In principle, further spatial directions could be covered metrologically by suitable superimposition of further laser beams having other wavelengths.

If a particle, such as, for example, a cavitation bubble 3, moves through the focus region 2, it scatters or reflects the light of the stationary light field in doing so. The scattered light produced is conveyed by the optical unit 10 over the reverse optical path, i.e. through the second lens 11, the mirror 13, the first lens 14 and the optical fiber bundle 15, to the facilities 20 outside the body. There, a module 22 that contains, inter alia, photomultipliers (secondary electron multipliers) records the variation in the intensity I of the back-scattered light against time t. If a particle moves through the focus region 2 with a certain velocity (vₓ, v_{y}, v_{z}) and consecutively traverses the intensity maxima and minima of the stationary light field, this is manifested in the measured intensity I of the scattered light by periodic fluctuations. The movement velocity of the particle in the direction of the modulations of the stationary light field can be inferred from the spacing of said fluctuations. Since such an analysis can be performed independently for the two wavelengths λ₁ and λ₂, the velocity components vₓ, v_{y} of a small cavitation bubble 3 moving through the focus region 2 can consequently be determined. Alternatively, the movement of the small cavitation bubble 3 could also take place (without additional lasers) on the basis of the sonoluminescence.

The wavelength λ_{K}, λ₁ and λ₂ of the participating lasers should, on the one hand, be sufficiently different in order to be able to distinguish them spectrally and, if necessary, separate them. On the other hand, they should not be large enough to disturb the chromatic effects of the optics. Suitable spectral filters in the facilities outside the body should prevent crosstalk occurring between the light beams of different origins. Furthermore, an adaptation to the refractive indexes of the serum and the blood particles can be undertaken if the measurements are disturbed by high scattering rate.

The radial or z-component of the movement of a small cavitation bubble 3 is measured in the device shown with the aid of the Doppler shift. For this purpose, the difference between the wavelength of the light (λ₁ or λ₂) injected is compared with the wavelength of the elastically back-scattered (reflected) light in a Doppler shift module 21 comprising a frequency analyzer, in which process the desired velocity component v_{z} can be inferred according to the Doppler principle from the differential wavelength Δλ.

As already mentioned, the focus region 2 can be systematically displaced in the lumen of the vessel in order to scan it. If the focus region 2 reaches the vessel wall 1 (or other structures having altered material properties) in doing so, a sudden and significant change in the back-scattered light occurs. In particular, the intensity of the back-scattered light may increase as a result of the reflection at the vessel wall 1. Furthermore, fluorescence processes can be excited in the vessel wall that result in the occurrence of fluorescence light of characteristic wavelength. As a result of the changes described, the evaluation facility 20 outside the body can detect when the focus region 2 reaches the vessel wall 1. This information can then be evaluated for different purposes, and specifically, in particular for:

A measurement of the vessel cross section or generally of the vessel structure, there being no restrictions in regard to the shape of the vessel.

An automatic switching-off of the cavitation light laser 30 in order to prevent damage to the tissue in and behind the vessel wall 1 as a result of high-power cavitation light. If the focus region 2 again enters the interior of the vessel lumen after continuing its scanning movement, the cavitation light laser source 30 can be switched on again.

The controlled initiation of processes or performance of measurements at the vessel wall 1. For example, knowledge of the flow conditions in the vicinity of the vessel wall is particularly important for estimating the risk of deposit formation. Furthermore, a known position of the focusing region 2 at the vessel wall can be utilized to undertake a local activation of drugs in a controlled (laser-induced) manner.

If the contact of the vessel wall 1 by the focus region 2 is to be detected as a result of fluorescence light occurring, a spectrometer should be provided in the analytical facility 20. In this connection, the information contained in the spectrum may also be used quite generally for a chemical or molecular, spatially resolved analysis of the vessel lumen and also of the surrounding tissue. For example, the concentration of certain drugs can be determined in a spatially resolved way from the fluorescent light characteristic thereof. A chemical characterization of the tissue may furthermore also be used to investigate deposits or to image the intestinal tissue.

1. A device for measuring flow in a fluid, comprising:
a) a cavitation unit (10, 30) for generating cavitation bubbles (3) in the fluid;
b) a particle-measuring unit (10, 20) for detecting the movement of cavitation bubbles (3) generated by the cavitation unit.

2. A device as claimed in claim 1, characterized in that the cavitation unit comprises a cavitation light laser source (30) and/or an ultrasonic source.

3. A device as claimed in claim 1, characterized in that the particle-measuring unit (10, 20) is designed to measure particle movement with the aid of phase-Doppler anemometry (22) and/or Doppler shift (21).

4. A device as claimed in claim 1, characterized in that the particle-measuring unit is designed to determine particle movement from the light emitted by the particles (3).

5. A facility for invasive intervention, comprising a catheter (16) having an optical unit (10) that is disposed at the catheter tip and that can receive light selectively from a focus region (2) situated outside the catheter and/or beam it into the focus region (2), in which process the radial position of the focus region (2) can be adjusted externally.

6. A facility as claimed in claim 5, characterized in that the optical unit (10) can be rotated around the catheter axis relative to the catheter (16).

7. A facility as claimed in claim 5, characterized in that the catheter (16) comprises a bundle (15) of optical waveguides that connects the optical unit (10) to the beginning of the catheter.

8. A facility as claimed in claim 5, characterized in that it comprises a scanning unit (20) that is designed to vary the position of the focus region (2) systematically and to analyze light picked up by the optical unit (10) from the respective focus region (2) with regard to characteristic properties of the focus area.

9. A facility as claimed in claim 5, comprises in that it comprises a spectrometer for the spectral analysis of light picked up from the focus region (2).

10. A facility as claimed in claim 5, characterized in that it comprises a particle-measuring unit (20) that is designed to generate a modulated light field for phase-Doppler anemometry in the focus region (2) via the optical unit (10).

11. A facility as claimed in claim 5, characterized in that it comprises an activation unit that is designed to inject light via the optical unit (10) into the focus region (2) in order to initiate local processes therein as a result of interaction with matter.

12. A facility as claimed in claim 11, characterized in that the activation unit (20) comprises a cavitation light laser source (30) and is designed to generate cavitation bubbles (3) in the focus region (2).

13. A method of measuring flow in a fluid, wherein cavitation bubbles (3) are generated in the fluid and the movement of the cavitation bubbles (3) is observed.

14. A method of determining the position of a vessel wall (1), wherein light is picked up from a focus region (2) that is continuously displaced in the vessel and a qualitative change in the light picked up is detected.
